(19) United States
(12) Patent Application Publication
(10) Pub. No.: US 2023/0064920 A1
(43) Pub. Date: Mar. 2, 2023

(54) PROCESS OF PREPARING ARACHIDONOYLETHANOLAMINE ANALOGUES

(71) Applicant: TRAVECTA THERAPEUTICS PTE. LTD., Singapore (SG)

(72) Inventors: Mahmood AHMED, Singapore (SG); Sing Yeung Frankie MAK, Singapore (SG); Ravi Kumar PUTHUKANOORI, Hyderabad (IN); Sampath Kumar NANGUNOORI, Hyderabad (IN); Ramana Murthy Venkata APPALA, Hyderabad (IN); Narendar VENNU, Hyderabad (IN); Filippo ROMOLI, Firenze (IT); Giovanni MARRAS, Verona (IT)

(73) Assignee: TRAVECTA THERAPEUTICS PTE. LTD., Singapore (SG)

(21) Appl. No.: 17/788,119

(22) PCT Filed: Nov. 20, 2020
(86) PCT No.: PCT/EP2020/082861

§ 371 Date: Jun. 22, 2022

Related U.S. Application Data
(60) Provisional application No. 62/938,502, filed on Nov. 21, 2019.

(51) Int. Cl. C07F 9/10; C07D 207/46; B01J 23/44; B01J 21/18

(52) U.S. Cl. CPC C07F 9/10; C07D 207/46; B01J 23/44; B01J 21/18

(57) ABSTRACT

The present application provides new processes for preparing arachidonoylethanolamine analogues. Intermediates useful for preparing the compounds are also provided.

### CROSS-REFERENCE TO RELATED APPLICATIONS

This application claims the benefit of U.S. Provisional Application Ser. No. 62/938,502, filed on Nov. 21, 2019, the disclosure of which is incorporated herein by reference in its entirety.

### TECHNICAL FIELD

The present application provides new processes for preparing arachidonoylethanolamine analogues.

### BACKGROUND

The endocannabinoid (eCB) system has been implicated in a variety of processes including cell signaling, memory encoding, compensatory mechanisms, and immunosuppressant and anti-inflammatory responses. The eCB system comprises at least two receptors: the CB1 cannabinoid receptor, widely distributed in the brain and present in some peripheral organs, and the CB2 receptor, found principally in the periphery and immune systems and in some regions of the brain. The endogenous agonists of these receptors are the endogenous cannabinoids (eCBs), a family of lipids comprising anandamide (AEA) and 2-arachidonyl glycerol (2-AG), as well as other closely related compounds (see e.g., Piomelli, Nat. Rev. Neurosci. 2003, 4(11), 873).

### SUMMARY

The present application provides, inter alia, processes of preparing a compound of Formula I:

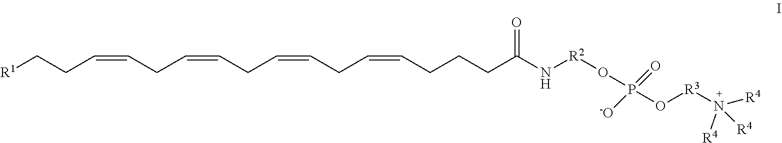

or pharmaceutically acceptable salts thereof, wherein constituent members are defined herein.

The present application further provides intermediates (e.g., Intermediate 1 as described herein) and salts thereof which are useful for the preparation of compounds of Formula I, or pharmaceutically acceptable salts thereof.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### DETAILED DESCRIPTION

The duration of in vivo CB1 and/or CB2 receptor modulation by AEA and 2-AG is relatively short, presumably due to a rapid inactivation process involving endocannabinoid deactivating proteins, with AEA and 2-AG predominantly hydrolyzed by Fatty Acid Amide Hydrolase (FAAH) and monoacylglycerol lipase (MAGL), respectively. FAAH and MAGL are serine hydrolases and their inhibition is known to increase the level of endogenous cannabinoid ligands, including AEA and 2-AG. The increased level of activation of the cannabinoid receptors resulting from increased levels of AEA and/or 2-AG has shown analgesic effect in acute and chronic models of pain, as well as a number of other animal models (e.g., depression, anxiety, inflammation, brain trauma, multiple sclerosis, cancer, and glaucoma) (see e.g., Nomura, Life Sci. 2013, 92(8-9), 492; and Mallet, Int. J. Clin. Pharmacol. Ther. 2016; 54(7), 498).

The present application provides new processes for preparing compounds that increase the levels of endogenous cannabinoid ligands in a subject in need thereof.

Accordingly, the present application provides processes of preparing a compound of Formula I:

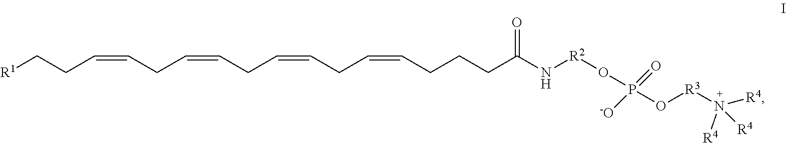

or a pharmaceutically acceptable salt thereof, wherein:

R¹ is C₁₋₁₀ alkyl;

R² is C₁₋₆ alkylene;

R³ is C₁₋₆ alkylene; and

each R⁴ is independently selected from the group consisting of H and C₁₋₆ alkyl.

In some embodiments, the process comprises reacting a compound of Formula II:

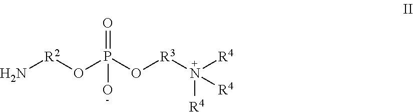

or a salt thereof, with a compound of Formula III:

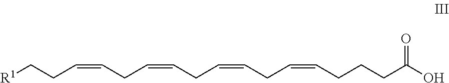

or a salt thereof, in the presence of a first base and an amine coupling agent, wherein:

R¹ is C₁₋₁₀ alkyl;

R² is C₁₋₆ alkylene;

R³ is C₁₋₆ alkylene; and

each R⁴ is independently selected from the group consisting of H and C₁₋₆ alkyl.

In some embodiments, the first base is an amine base. In some embodiments, the first base is a tri(C₁₋₆ alkyl) amine base. In some embodiments, the first base is N,N-diisopropylethylamine.

In some embodiments, the amine coupling agent is 1-[bis(dimethylamino)methylene]-1H-1,2,3-triazolo[4,5-b]pyridinium 3-oxide hexafluorophosphate (HATU).

In some embodiments, the reacting comprises mixing the amine coupling agent with the compound of Formula III to afford a second mixture; and adding the first base and the compound of Formula II, sequentially, to the second mixture.

In some embodiments, the reacting comprises mixing the amine coupling agent with the compound of Formula III to afford a second mixture; and adding the compound of Formula II and then the first base, sequentially, to the second mixture.

In some embodiments, the reacting comprises mixing the amine coupling agent with the compound of Formula III to afford a second mixture; and adding the first base and the compound of Formula II to the second mixture.

In some embodiments, about 1 to about 5 equivalents (e.g., about 1 equivalent, about 2 equivalents, about 3 equivalents, about 4 equivalents, or about 5 equivalents) of the compound of Formula II is used based on 1 equivalent of the compound of Formula III.

In some embodiments, the reacting is performed at a temperature of from about 20° C. to about 30° C. (i.e., about room temperature, for example about 20° C., about 21° C., about 22° C., about 23° C., about 24° C., about 25° C., about 26° C., about 27° C., about 28° C., about 29° C., or about 30° C.).

In some embodiments, the reacting is performed for about 4 hours to about 40 hours, e.g., about 4 hours, about 6 hours, about 8 hours, about 10 hours, about 12 hours, about 14 hours, about 16 hours, about 18 hours, about 20 hours, about 24 hours, about 28 hours, about 30 hours, about 32 hours, about 36 hours, or about 40 hours.

In some embodiments, the reacting is performed in the presence of a first solvent component. In some embodiments, the first solvent component comprises dimethylformamide.

In some embodiments, the process further comprises separating and/or isolating the by-products and/or unreacted compounds (i.e., unreacted compound of Formula II and unreacted compound of Formula III) from the desired product (i.e., compound of Formula I, or a pharmaceutically acceptable salt thereof). In some embodiments, the process further comprises isolating the compound of Formula I, or a pharmaceutically acceptable salt thereof.

In some embodiments, the isolating is performed by diluting the reaction mixture comprising the compound of Formula I with a fifth solvent component. In some embodiments, the isolating is performed by concentrating the reaction mixture comprising the compound of Formula I by removing the solvent. In some embodiments, the isolating comprises concentrating the reaction mixture comprising the compound of Formula I, or a pharmaceutically acceptable salt thereof, followed by a solvent swap (or solvent exchange) with a fifth solvent component. In some embodiments, the isolating may be repeated (e.g., once, twice, three times, and so forth) so as to recover more of the compound of Formula I, or a pharmaceutically acceptable salt thereof.

In some embodiments, the fifth solvent component is isopropylether (IPE) or n-heptane. In some embodiments, the fifth solvent component comprises isopropylether (IPE). In some embodiments, the fifth solvent component comprises n-heptane.

In some embodiments, the compound of Formula II is prepared according to a process comprising deprotecting a compound of Formula IV:

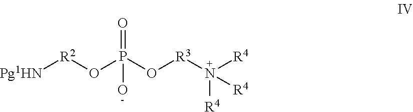

or a salt thereof, wherein:

Pg¹ is an amine protecting group, wherein:

R² is C₁₋₆ alkylene;

R³ is C₁₋₆ alkylene; and

each R⁴ is independently selected from the group consisting of H and C₁₋₆ alkyl.

In some embodiments, Pg¹ is benzyloxycarbonyl (Cbz).

In some embodiments, the deprotecting comprises reacting the compound of Formula IV with hydrogen gas in the presence of a hydrogenation catalyst. In some embodiments, the hydrogenation catalyst comprises palladium on carbon. In some embodiments, the compound of Formula IV is reacted with the hydrogen gas, wherein the pressure of the hydrogen gas is from about 1 bar to about 20 bar, e.g., about 1 bar, about 2 bar, about 3 bar, about 4 bar, about 5 bar, about 6 bar, about 7 bar, about 8 bar, about 9 bar, about 10 bar, about 12 bar, about 14 bar, about 16 bar, about 18 bar, or about 20 bar.

In some embodiments, the deprotecting is performed at a temperature of from about 20° C. to about 30° C. (i.e., about room temperature, e.g., about 20° C., about 21° C., about 22° C., about 23° C., about 24° C., about 25° C., about 26° C., about 27° C., about 28° C., about 29° C., or about 30° C.).

In some embodiments, the deprotecting is performed for about 4 hours to about 40 hours, e.g., about 4 hours, about 6 hours, about 8 hours, about 10 hours, about 12 hours, about 14 hours, about 16 hours, about 18 hours, about 20 hours, about 24 hours, about 28 hours, about 30 hours, about 32 hours, about 36 hours, or about 40 hours.

In some embodiments, the deprotecting is performed in the presence of a second solvent component. In some embodiments, the second solvent component comprises water or a C₁₋₃ alcohol (e.g., methanol (MeOH), ethanol (EtOH), n-propanol, isopropanol, or mixures thereof). In some embodiments, the second solvent component comprises methanol.

In some embodiments, the compound of Formula IV is prepared according to a process comprising:
i) reacting a compound of Formula V:

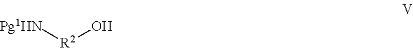

or a salt thereof, with phosphoryl trihalide in the presence of a second base to afford a first mixture; and
ii) reacting the first mixture with a compound of Formula VI:

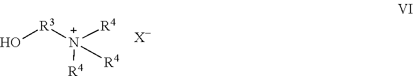

in the presence of a third base, wherein:

X⁻ is an anion;

Pg¹ is an amine protecting group;

R² is C₁₋₆ alkylene;

R³ is C₁₋₆ alkylene; and

each R⁴ is independently selected from the group consisting of H and C₁₋₆ alkyl.

In some embodiments, Pg¹ is benzyloxycarbonyl (Cbz).

In some embodiments, X⁻ is tosylate.

In some embodiments, the second base is an amine base. In some embodiments, the second base is a tri(C₁₋₆ alkyl) amine base. In some embodiments, the second base is triethylamine.

In some embodiments, the reacting of step i) is performed at a temperature of from about −20° C. to about 25° C. (e.g., about −20° C., about −15° C., about −10° C., about −5° C., about 0° C., about 5° C., about 10° C., about 15° C., about 20° C. or about 25° C.). In some embodiments, the reacting of step i) is performed at a temperature of from about 0° C. to about 25° C. In some embodiments, the reacting of step i) is performed for about 10 minutes to about 6 hours, e.g., about 10 minutes, about 30 minutes, about 1 hour, about 2 hours, about 3 hours, about 4 hours, about 5 hours, or about 6 hours.

In some embodiments, the reacting of step i) comprises mixing the compound of Formula V, or a salt thereof, and the second base to form a mixture, and reacting the mixture with phosphoryl trihalide.

In some embodiments, the reacting of step i) comprises:
a) adding the second base and phosphoryl trihalide, sequentially, to the compound of Formula V, or a salt thereof, or
b) adding the phosphoryl trihalide and second base, sequentially, to the compound of Formula V, or a salt thereof, wherein the sequential addition is performed at a temperature of from about −20° C. to about 10° C. (e.g., about −20° C., about −15° C., about −10° C., about −5° C., about 0° C., about 5° C., or about 10° C.).

In some embodiments, step i) is performed in the presence of a third solvent component. In some embodiments, the third solvent component comprises chloroform or dichloromethane. In some embodiments, the third solvent component comprises chloroform. In some embodiments, the third solvent component comprises dichloromethane.

In some embodiments, the third base is an amine base. In some embodiments, the third base is pyridine.

In some embodiments, step ii) is performed at a temperature of from about −10° C. to about 25° C. (e.g., about −10° C., about −5° C., about 0° C., about 5° C., about 10° C., about 15° C., about 20° C. or about 25° C.). In some embodiments, step ii) is performed for the duration of from about 4 hours to about 20 hours, e.g., about 4 hours, about 6 hours, about 8 hours, about 10 hours, about 12 hours, about 14 hours, about 16 hours, about 18 hours or about 20 hours.

In some embodiments, step i) and step ii) are advantageously performed as a single pot reaction (e.g., without substantially isolating the product of step i)).

In some embodiments, the compound of Formula V is prepared according to a process comprising reacting a compound of Formula VII:

Pg¹—X¹ VII

or a salt thereof, with a compound of Formula IX:

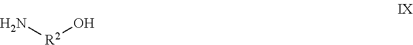

in the presence of a fourth base, wherein:

Pg¹ is an amine protecting group;

X¹ is halo; and

R² is C₁₋₆ alkylene.

In some embodiments, X¹ is chloro.

In some embodiments, the reacting is performed in the presence of a fourth solvent component.

In some embodiments, the fourth solvent component is dichloromethane.

In some embodiments, the reacting comprises adding the compound of Formula IX to the compound of Formula VII in the presence of the fourth solvent component. In some embodiments, the reacting comprises adding the compound of Formula VII to the compound of Formula IX in the presence of the fourth solvent component. In some embodiments, the addition step comprises a drop wise addition of the reagent.

In some embodiments, the reacting is performed at a temperature of from about −10° C. to about 10° C. (e.g., about −10° C., about −5° C., about 0° C., about 5° C. or about 10° C.).

In some embodiments, the reacting is performed for about 1 hour to about 10 hours, e.g., about 1 hour, about 2 hours, about 3 hours, about 4 hours, about 5 hours, about 6 hours, about 7 hours, about 8 hours, about 9 hours, or about 10 hours.

In some embodiments, R¹ is C₁₋₆ alkyl. In some embodiments, R¹ is propyl.

In some embodiments, R² is ethylene.

In some embodiments, R³ is ethylene.

In some embodiments, each R⁴ is an independently selected C₁₋₆ alkyl. In some embodiments, each R⁴ is methyl. In some embodiments, each R⁴ is H.

In some embodiments, the compound of Formula I is a compound of Formula Ia:

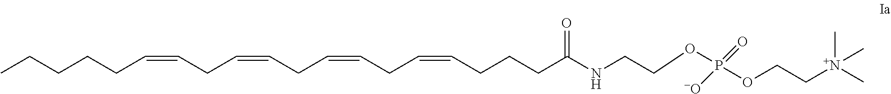

or a pharmaceutically acceptable salt thereof.

The present application further provides processes of preparing a compound of Formula Ia:

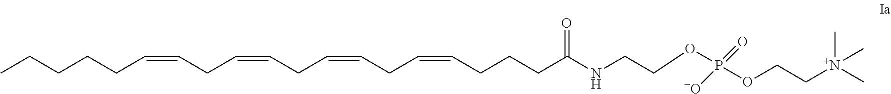

or a pharmaceutically acceptable salt thereof.

In some embodiments, the process of preparing the compound of Formula Ia comprises reacting a compound of Formula IIa:

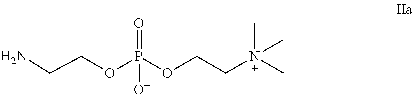

or a salt thereof, with a compound of Formula IIIa:

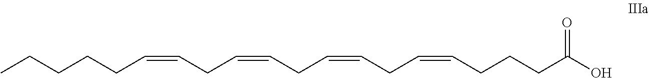 [SMILES: CCCCC/C=C\C/C=C\C/C=C\C/C=C\CCCC(=O)O]

or a salt thereof, in the presence of N,N-diisopropylethylamine and [bis(dimethylamino)methylene]-1H-1,2,3-triazolo[4,5-b]pyridinium 3-oxide hexafluorophosphate (HATU).

In some embodiments, the process of preparing the compound of Formula Ia, or a pharmaceutically acceptable salt thereof, further comprises isolating the compound of Formula Ia, or a pharmaceutically acceptable salt thereof.

In some embodiments, the isolating comprises diluting the reaction mixture comprising the compound of Formula Ia, or a pharmaceutically acceptable salt thereof, with a fifth solvent component.

In some embodiments, the isolating comprises concentrating the reaction mixture comprising the compound of Formula Ia, or a pharmaceutically acceptable salt thereof, and performing a solvent swap with a fifth solvent component, thereby isolating the compound of Formula Ia, or a pharmaceutically acceptable salt thereof.

In some embodiments, the solvent swap is repeated one to four times. In some embodiments, the fifth solvent comprises isopropylether or n-heptane.

The present application further provides processes of preparing a compound of Formula I:

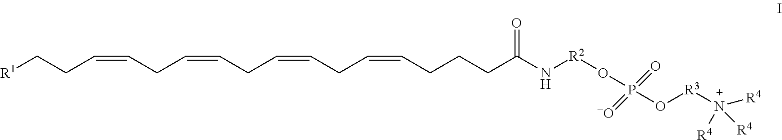

or a pharmaceutically acceptable salt thereof, comprising reacting a compound of Formula II:

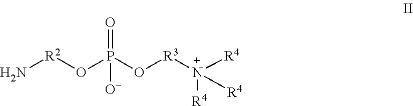

with a compound of Formula X:

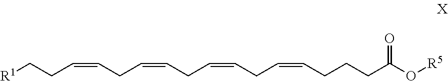

or a salt thereof, in the presence of a first base, wherein:

R¹ is C₁₋₁₀ alkyl;

R² is C₁₋₆ alkylene;

R³ is C₁₋₆ alkylene;

each R⁴ is independently selected from the group consisting of H and C₁₋₆ alkyl; and

R⁵ is a carboxylic acid activating group.

In some embodiments, the first base is an amine base. In some embodiments, the first base is tri(C₁₋₆ alkyl) amine base. In some embodiments, the first base is triethylamine.

In some embodiments, R¹ is C₁₋₆ alkyl. In some embodiments, R¹ is propyl.

In some embodiments, R² is ethylene.

In some embodiments, R³ is ethylene.

In some embodiments, each R⁴ is an independently selected C₁₋₆ alkyl. In some embodiments, each R⁴ is methyl. In some embodiments, each R⁴ is H.

In some embodiments, R⁵ is 2,5-dioxopyrrolidin-1-yl.

In some embodiments, about 1 to about 2 equivalents of the compound of Formula II is used based on 1 equivalent of the compound of Formula III.

In some embodiments, the reacting is performed at a temperature of from about 20° C. to about 30° C. (i.e., about room temperature, e.g., about 20° C., about 21° C., about 22° C., about 23° C., about 24° C., about 25° C., about 26° C., about 27° C., about 28° C., about 29° C., or about 30° C.).

In some embodiments, the reacting is performed from about 4 hours to about 40 hours, e.g., about 4 hours, about 6 hours, about 8 hours, about 10 hours, about 12 hours, about 14 hours, about 16 hours, about 18 hours, about 20 hours, about 24 hours, about 28 hours, about 30 hours, about 32 hours, about 36 hours, or about 40 hours.

In some embodiments, the reacting is performed in the presence of a first solvent component. In some embodiments, the first solvent component comprises dimethylformamide.

In some embodiments, the compound of Formula X is prepared according to a process comprising reacting a compound of Formula XI:

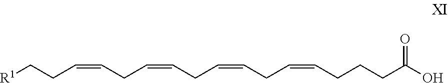

or a salt thereof, with a carboxylic acid activating agent in the presence of a 0-N coupling agent, wherein R¹ is C₁₋₁₀ alkyl.

In some embodiments, the carboxylic acid activating agent is N-hydroxysuccinimide (NHS).

In some embodiments, the O—N coupling agent is N,N′-dicyclohexylcarbodiimide (DCC).

In some embodiments, the reacting is performed at a temperature of from about 20° C. to about 30° C. (i.e., about room temperature, e.g., about 20° C., about 21° C., about 22° C., about 23° C., about 24° C., about 25° C., about 26° C., about 27° C., about 28° C., about 29° C., or about 30° C.).

In some embodiments, the reacting is performed for about 4 hours to about 40 hours, e.g., about 4 hours, about 6 hours, about 8 hours, about 10 hours, about 12 hours, about 14 hours, about 16 hours, about 18 hours, about 20 hours, about 24 hours, about 28 hours, about 30 hours, about 32 hours, about 36 hours, or about 40 hours.

In some embodiments, the reacting is performed in the presence of a second solvent component. In some embodiments, the second solvent component comprises ethyl acetate.

The present application further provides processes of preparing a compound of Formula Ia:

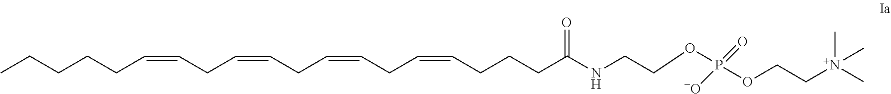

or a pharmaceutically acceptable salt thereof, comprising reacting a compound of Formula IIa:

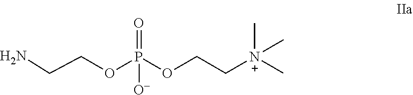

or a salt thereof, with a compound of Formula Xa:

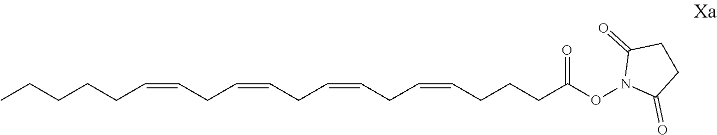 [SMILES: CCCCC/C=C\C/C=C\C/C=C\C/C=C\CCCC(=O)ON1C(=O)CCC1=O]

or a salt thereof, in the presence of triethylamine.

The present application further provides a compound of Formula Xa:

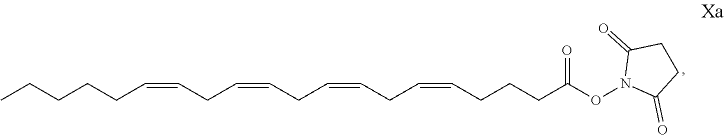 [SMILES: CCCCC/C=C\C/C=C\C/C=C\C/C=C\CCCC(=O)ON1C(=O)CCC1=O]

or a salt thereof.

It is appreciated that certain features of the invention, which are, for clarity, described in the context of separate embodiments, can also be provided in combination in a single embodiment (while the embodiments are intended to be combined as if written in multiply dependent form). Conversely, various features of the invention which are, for brevity, described in the context of a single embodiment, can also be provided separately or in any suitable subcombination.

At various places in the present specification, substituents of compounds of the invention are disclosed in groups or in ranges. It is specifically intended that the invention include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

As used herein, the term “Cₙ₋ₘ alkyl”, employed alone or in combination with other terms, refers to a saturated hydrocarbon group that may be straight-chain or branched, having n to m carbon atoms. In some embodiments, the alkyl group contains 1 to 6, 1 to 4 or 1 to 3 carbon atoms. Examples of alkyl moieties include, but are not limited to, chemical groups such as methyl, ethyl, n-propyl, isopropyl, n-butyl, isobutyl, sec-butyl, tert-butyl, n-pentyl, 2-methyl-1-butyl, 3-pentyl, n-hexyl, 1,2,2-trimethylpropyl, and the like.

As used herein, the term “amine base” refers to a mono-substituted amine group (i.e., primary amine base), di-substituted amine group (i.e., secondary amine base), or a tri-substituted amine group (i.e., tertiary amine base). Exemplary amine bases include, bt are not limited to, methyl amine, ethyl amine, propyl amine, butyl amine, dimethylamine, diethylamine, dipropylamine, dibutylamine, trimethylamine, triethylamine, tripropylamine, triisopropylamine, tributylamine, tri-tert-butylamine, N,N-dimethylethanamine, N,N-diisopropylethylamine, pyridine, and the like.

As used herein, “halo”, refers to fluoro, chloro, bromo, and iodo. In some embodiments, the halo is chloro.

As used herein the term, “hydrogenation catalyst” refers to a metal (e.g., platinum, palladium, nickel, ruthenium or rhodium) catalyst suitable to catalyze a hydrogenation reaction (i.e., reaction of a compound with hydrogen gas). Exemplary hydrogenation catalysts include, but are not limited to, palladium on carbon, Lindlar's catalyst (palladium deposited on calcium carbonate or barium sulfate), Wilkinson's catalyst, HRuCl(PPh₃)₃, RhCl(PPh₃)₃, [Rh(COD)Cl]₂, [Ir(COD)(PMePh₂)₂]⁺, [Rh(1,5-cyclooctadiene)(PPh₃)₂]⁺, PtO₂ (Adam's catalyst), palladium black, and the like. In some embodiments, the hydrogenation catalyst is palladium on carbon (Pd/C).

Preparation of the compounds described herein can involve the protection and deprotection of various chemical groups (e.g., protection and deprotection of amine groups). The need for protection and deprotection, and the selection of appropriate protecting groups, can be readily determined by one skilled in the art. The chemistry of protecting groups can be found, for example, in Wuts and Greene, Protective Groups in Organic Synthesis, 4th ed., John Wiley & Sons: New Jersey, (2007), the disclosure of which is incorporated herein by reference in its entirety. Adjustments to the protecting groups and formation and cleavage methods described herein may be adjusted as necessary in light of the various substituents.

As used herein, the term “Pg” refers to a protecting group (e.g., an amine protecting groups). Exemplary amine protecting groups include, but are not limited to, benzyloxycarbonyl (Cbz), 2,2,2-trichloroethoxycarbonyl (Troc), 2-(trimethylsilyl)ethoxycarbonyl (Teoc), 2-(4-trifluoromethylphenylsulfonyl)ethoxycarbonyl (Tsc), t-butoxycarbonyl (BOC), 1-adamantyloxycarbonyl (Adoc), 2-adamantylcarbonyl (2-Adoc), 2,4-dimethylpent-3-yloxycarbonyl (Doc), cyclohexyloxycarbonyl (Hoc), 1,1-dimethyl-2,2,2-trichloroethoxycarbonyl (TcBOC), vinyl, 2-chloroethyl, 2-phenylsulfonylethyl, allyl, benzyl, 2-nitrobenzyl, 4-nitrobenzyl, diphenyl-4-pyridylmethyl, N′,N′-dimethylhydrazinyl, methoxymethyl, t-butoxymethyl (Bum), benzyloxymethyl (BOM), or 2-tetrahydropyranyl (THP), tri(C₁₋₄ alkyl)silyl (e.g., tri(isopropyl)silyl), 1,1-diethoxymethyl, or N-pivaloyloxymethyl (POM). In some embodiments, the protecting group is benzyloxycarbonyl (Cbz).

As used herein, the terms “deprotecting” or “deprotection conditions” refer to conditions suitable to cleave a protecting group (e.g., an amine protecting group). In some embodiments, deprotection conditions may include cleavage of a protecting group in the presence of a strong acid, in the presence of a strong base, in the presence of a reducing agent, or in the presence of an oxidizing agent. Deprotection of an amine protecting group can be accomplished by methods known in the art for the removal of particular protecting groups for amines, such as those in Wuts and Greene, Protective Groups in Organic Synthesis, 4th ed., John Wiley & Sons: New Jersey, pages 696-887 (and, in particular, pages 872-887) (2007), the disclosure of which is incorporated herein by reference in its entirety.

All compounds, and pharmaceutically acceptable salts thereof, can be found together with other substances such as water and solvents (e.g., hydrates and solvates) or can be isolated.

In some embodiments, the compounds of the invention, or salts thereof, are substantially isolated. By “substantially isolated” is meant that the compound is at least partially or substantially separated from the environment in which it was formed or detected. Partial separation can include, for example, a composition enriched in the compounds of the invention. Substantial separation can include compositions containing at least about 50%, at least about 60%, at least about 70%, at least about 80%, at least about 90%, at least about 95%, at least about 97%, or at least about 99% by weight of the compounds of the invention, or salt thereof. Methods for isolating compounds and their salts are routine in the art.

As used herein, the term “reacting” is used as known in the art and generally refers to the bringing together of chemical reagents in such a manner so as to allow their interaction at the molecular level to achieve a chemical or physical transformation. In some embodiments, the reacting involves two reagents, wherein one or more equivalents of second reagent are used with respect to the first reagent. The reacting steps of the processes described herein can be conducted for a time and under conditions suitable for preparing the identified product.

The reactions of the processes described herein can be carried out in suitable solvents which can be readily selected by one of skill in the art of organic synthesis. Suitable solvents can be substantially nonreactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected. In some embodiments, reactions can be carried out in the absence of solvent, such as when at least one of the reagents is a liquid or gas.

As used herein, a “solvent component” refers to one solvent or a mixture of two or more solvents.

As used herein, “second”, “third,” “fourth”, etc. as a prefix to the phrase “solvent component” or “amine base” is used to differentiate the solvent component or amine base from other solvent components or amine bases used in earlier or later steps of the process and does not indicate that multiple solvents or bases must be present.

Exemplary halogenated solvents include, but are not limited to, carbon tetrachloride, chloroform, dichloromethane, and the like.

Exemplary ether solvents include, but are not limited to, tetrahydrofuran, 1,3-dioxane, 1,4-dioxane, furan, diethyl ether, ethylene glycol dimethyl ether, ethylene glycol diethyl ether, diethylene glycol dimethyl ether (diglyme), t-butyl methyl ether, and the like.

Exemplary protic solvents include, but are not limited to, water, methanol (MeOH), ethanol (EtOH), ethylene glycol, 1-propanol, 2-propanol, 1-butanol, 2-butanol, i-butyl alcohol, t-butyl alcohol, and the like.

Exemplary aprotic solvents include, but are not limited to, tetrahydrofuran (THF), N,N-dimethylformamide (DMF), N,N-dimethylacetamide (DMA), 1,3-dimethyl-3,4,5,6-tetrahydro-2(1H)-pyrimidinone (DMPU), 1,3-dimethyl-2-imidazolidinone (DMI), N-methylpyrrolidinone (NMP), formamide, N-methylacetamide, N-methylformamide, acetonitrile (ACN), dimethyl sulfoxide (DMSO), acetone, ethyl methyl ketone, ethyl acetate (EtOAc), and the like.

Exemplary hydrocarbon solvents include, but are not limited to, benzene, cyclohexane, pentane, hexane, toluene, and the like.

The reactions of the processes described herein can be carried out in air or under an inert atmosphere. Typically, reactions containing reagents or products that are substantially reactive with air can be carried out using air-sensitive synthetic techniques that are well known to the skilled artisan.

In some embodiments, preparation of compounds can involve the addition of acids or bases to affect, for example, catalysis of a desired reaction or formation of salt forms such as acid addition salts.

Exemplary inorganic acids include, but are not limited to, hydrochloric acid, hydrobromic acid, sulfuric acid, phosphoric acid, nitric acid, and the like. Exemplary organic acids include, but are not limited to, formic acid, acetic acid, propionic acid, butanoic acid, benzoic acid, methanesulfonic acid, p-toluenesulfonic acid, benzenesulfonic acid, trifluoroacetic acid (TFA), and the like.

Exemplary bases include, but are not limited to, lithium hydroxide, sodium hydroxide, potassium hydroxide, lithium carbonate, sodium carbonate, potassium carbonate, and sodium bicarbonate. Exemplary strong bases include, but are not limited to, hydroxide, alkoxides, metal amides, metal hydrides, metal dialkylamides, and arylamines, wherein; alkoxides include lithium, sodium and potassium salts of methyl, ethyl and t-butyl oxides; metal amides include sodium amide, potassium amide and lithium amide; metal hydrides include sodium hydride, potassium hydride and lithium hydride; and metal dialkylamides include lithium, sodium, and potassium salts of methyl, ethyl, n-propyl, i-propyl, n-butyl, t-butyl, trimethylsilyl and cyclohexyl substituted amides.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms.

Resolution of racemic mixtures of compounds can be carried out by any of numerous methods known in the art. An example method includes fractional recrystallizaion using a chiral resolving acid which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids such as β-camphorsulfonic acid. Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine).

Suitable elution solvent composition can be determined by one skilled in the art.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Tautomeric forms can be in equilibrium or sterically locked into one form by appropriate substitution.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present application also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present application include the conventional non-toxic salts of the parent compound formed, for example, from non-toxic inorganic or organic acids. The pharmaceutically acceptable salts of the present application can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (ACN) are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977). Conventional methods for preparing salt forms are described, for example, in Handbook of Pharmaceutical Salts: Properties, Selection, and Use, Wiley-VCH, 2002.

Upon carrying out preparation of compounds according to the processes described herein, the usual isolation and purification operations such as concentration, filtration, extraction, solid-phase extraction, recrystallization, chromatography, and the like may be used, to isolate the desired products.

As used herein, the term “room temperature,” refers generally to a temperature (e.g., a reaction temperature) that is about the temperature of the room in which the reaction is carried out, for example, a temperature from about 20° C. to about 30° C.

The reactions of the processes described herein can be carried out at appropriate temperatures which can be readily determined by the skilled artisan. Reaction temperatures will depend on, for example, the melting and boiling points of the reagents and solvent, if present; the thermodynamics of the reaction (e.g., vigorously exothermic reactions may need to be carried out at reduced temperatures); and the kinetics of the reaction (e.g., a high activation energy barrier may need elevated temperatures).

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatographic methods such as high performance liquid chromatography (HPLC) or thin layer chromatography (TLC).

### Examples

The invention will be described in greater detail by way of specific examples.

The following examples are offered for illustrative purposes, and are not intended to limit the invention in any manner. Those of skill in the art will readily recognize a variety of noncritical parameters which can be changed or modified to yield essentially the same results. Analytical methods described throughout the Examples were performed according to the following procedures:

LC-MS-Method A

| Instrument | Shimadzu | |
|---|---|---|
| Injection volume | 3 | μL |
| Solvent A | ACN | |
| Solvent B | Water + 0.01% HCOOH | |
| Flow rate | 0.5 | mL/min |

| | |
|---|---|
| Temperature | Ambient |
| Column | LUNA-C18, |
| | 50 × 2.1 mm, 1.6 μm |

Solvent Gradient

| Time (min) | Solvent B[%] |
|---|---|
| 0 | 95 |
| 8 | 10 |
| 10 | 10 |

LC-MS-Method B

| Instrument | Agilent | |
|---|---|---|
| Injection volume | 1.6 | μL |
| Solvent A | Water + 0.1% HCOOH | |
| Solvent B | ACN | |
| Flow rate | 0.5 | mL/min |

| | |
|---|---|
| Temperature | Ambient |
| Column | LUNA OMEGA POLAR, |
| | 50 × 2.1 mm, 1.6 μm |

Solvent Gradient

| Time (min) | Solvent B[%] |
|---|---|
| 0 | 5 |
| 1.8 | 95 |
| 4.5 | 95 |

LC-MS-Method C

| Instrument | Shimadzu | |
|---|---|---|
| Injection volume | 3 | μL |
| Solvent A | ACN | |
| Solvent B | Water + 0.01% HCOOH | |
| Flow rate | 0.6 | mL/min |

| | |
|---|---|
| Temperature | Ambient |
| Column | Gemini-C18, |
| | 50 × 2.0 mm, 3.0 μm |

Solvent Gradient

| Time (min) | Solvent B[%] |
|---|---|
| 0.01 | 95 |
| 3 | 10 |
| 6 | 10 |

PREP-HPLC

| | |
|---|---|
| Instrument | Shimadzu |
| Injection volume | 80 mg per injection |
| Solvent A | ACN |
| Solvent B | Water + 0.01% HCOOH |
| Flow rate | 30 mL/min |
| Temperature | Ambient |
| Column | LUNA-C18, |
| | 250 × 21.2 mm, 1.6 μm |

Solvent Gradient

| Time (min) | Solvent B[%] |
|---|---|
| 0 | 80 |
| 10 | 50 |
| 22 | 50 |
| 35 | 90 |

Analytical HPLC Method

| Instrument | Agilent | |
|---|---|---|
| Injection volume | 10 | μL |
| Solvent A | Water + 0.01% TFA | |
| Solvent B | ACN | |
| Flow rate | 1.0 | mL/min |

| | |
|---|---|
| Temperature | Ambient |
| Column | Gemini-C18, |
| | 4.6 × 250 mm, 5 μm |

Solvent Gradient

| Time (min) | Solvent B[%] |
|---|---|
| 0 | 5 |
| 20 | 90 |
| 30 | 90 |

Flash Chromatography-Method A

| | |
|---|---|
| Instrument | Buchi |
| Injection volume | 1.0 g per injection |
| Solvent A | Water + 0.1% TFA |
| Solvent B | ACN |
| Flow rate | 25 mL/min |
| Temperature | Ambient |
| Column | Biotage-C18, |
| | 60 g Duo-100 Å 30 μm |

Solvent Gradient

| Time (min) | Solvent B[%] |
|---|---|
| 0 | 5 |
| 20 | 8 |
| 45 | 8 |

Flash Chromotography-Method B

| | |
|---|---|
| Instrument | Buchi |
| Injection volume | 2.0 g per injection |
| Solvent A | Water + 0.1% HCOOH |
| Solvent B | ACN |
| Flow rate | 30 mL/min |
| Temperature | Ambient |
| Column | Biotage-C18, |
| | 60 g Duo-100 Å 30 μm |

Solvent Gradient

| Time (min) | Solvent B[%] |
|---|---|
| 0 | 5 |
| 20 | 30 |
| 45 | 45 |

UPLC-MS generic Method

| Instrumental | |
|---|---|
| Parameter | Value |
| Column: | Kinetex 1.7 μm EVO C-18 100 A, 2.1 × 50 mm, |
| | column temperature 40° C. |
| Mobile Phase: | A: 0.1% TFA/water; B: ACN |
| Gradient: | 97% A to 99.9% B in 1.5 min, hold 99.9% B for |
| | 0.4 min, to 97% A in 0.1 min |
| Flow: | 1 mL/min |
| Ionization: | alternate Positive (ES+)/Negative Electrospray (ES−) |
| Scan Range: | both ES+ and ES− 100 to 1500 AMU |
| Scan Duration: | 0.10 seconds |

Analytical HPLC Method (40 min)

| Instrumental | |
|---|---|
| Parameter | Value |
| Column: | Phenomenex Gemini C-18, 4.6 × 250 mm, 5 μm |
| | column temperature 20° C. |
| Mobile Phase: | A: 0.1% Formic Acid/water; B: ACN |
| Gradient: | 0 min 5% B; 20 min 90% B, 30 min 90% B; 30.1 min |
| | 5% B; 40 min 5% B |
| Flow: | 1 mL/min |
| Detector: | UV @ 210 nm |

Preparation of Intermediate 1. 2-aminoethyl (2-(trimethylammonio)ethyl) phosphate

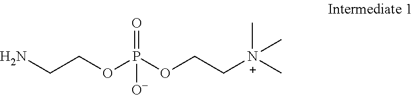

Step 1. benzyl (2-hydroxyethyl)carbamate

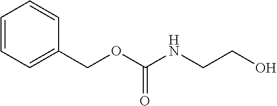 [SMILES: O=C(NCCO)OCc1ccccc1]

To a stirred solution of 2-aminoethan-1-ol (50 g, 0.819 mol, 1.0 eq.) in dichloromethane (DCM, 1.5 L) at 0° C. was added triethylamine, (Et3N, 137 mL, 0.983 mol, 1.2 eq.) dropwise. The reaction mixture was stirred for 10 minutes at 0° C. and after 10 min, benzyl carbonochloridate (Cbz-Cl; 50%; 302 mL, 1.064 mol, 1.2 eq) was added to the reaction mixture drop wise at 0° C. Then the reaction was stirred for 3 hours at 0° C. and monitored by thin layer chromatography (TLC). The reaction mixture was then quenched with water (500 mL) and extracted with DCM (3×500 mL). The total organic layer was dried over anhydrous Na₂SO₄, filtered, and concentrated to obtain crude compound (200 g), which was purified by silica gel column chromatography using gradient elution with 3% MeOH/DCM to afford benzyl (2-hydroxyethyl)carbamate (95 g) as a white solid. Mass [m/z]: 196.09 [M+H]⁺. Yield: 95 g (59.7%). HPLC Purity: 98% (Analytical HPLC method).

Step 2. 2-(((benzyloxy)carbonyl)amino)ethyl(2-(trimethylammonio)ethyl)phosphate

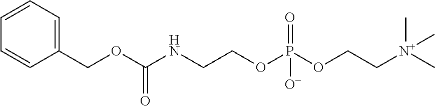 [SMILES: C[N+](C)(C)CCOP(=O)([O-])OCCNC(=O)OCc1ccccc1]

To a stirred solution of benzyl (2-hydroxyethyl)carbamate (5 g, 25.641 mmol, 1 eq.) in chloroform (100 mL) was added Et3N (5.5 mL, 38.461 mmol, 1.5 eq.) followed by phosphoryl trichloride (POCl₃, 2.65 mL, 28.205 mmol, 1.1 eq.) at −10° C. Then the reaction mixture was stirred for 1 hour at 25° C. and monitored by TLC. After 1 hour, pyridine (17.5 mL, 220.512 mmol, 8.6 eq.) and choline tosylate (10.55 g, 38.461 mmol, 1.5 eq) were added at −10° C. and the resulting mixture was stirred at 25° C. . After being stirred for 18 hours, the reaction mass was cooled to 0° C., quenched with water (20 mL), and extracted with DCM (3×100 mL). The aqueous layer was purified by flash chromatography [Column: Biotage-C18, 60 g Duo-100 Å 30 μm; Mobile phase: [ACN: Water+0.1% TFA]; B %: B %: 0-8%, 0-20 min/8% 20-45 min.] to afford 2-(((benzyloxy)carbonyl)amino)ethyl(2-(trimethylammonio)ethyl)phosphate (2.4 g) as a colorless liquid. Mass [m/z]: 361.01 [M+H]+. Yield: 2.4 g (26%). LC-MS Purity: 85% (LCMS Method B).

Step 3. 2-aminoethyl(2-(trimethylammonio)ethyl)phosphate (Intermediate 1)

To a stirred solution of 2-(((benzyloxy)carbonyl)amino)ethyl(2-(trimethylammonio)ethyl)phosphate (2.3 g, 0.638 mmol, 1.0 eq.) in isopropyl alcohol (or isopropanol, IPA; 20 mL) was added 10% Pd/C (50% wet; 500 mg) at 25° C. The reaction mixture was stirred under hydrogen pressure (from about 45 psi (or about 3.1 bar) to about 55 psi (or about 3.8 bar)) at 25° C. for 18 hours. The resulting mixture was filtered through a celite pad and washed with IPA. The filtrate was concentrated and dried under vacuum to yield 2-aminoethyl(2-(trimethylammonio)ethyl)phosphate (1.6 g) as a colorless liquid. The product was used in the following Examples without further purification. Mass [m/z]: 227.5 [M+H]+. Yield: 1.6 g (93%). HPLC Purity: 65% (Analytical HPLC method).

Alternative Preparation of Intermediate 1. 2-aminoethyl (2-(trimethylammonio)ethyl) phosphate

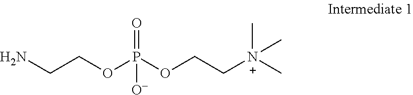

Step 1. benzyl (2-hydroxyethyl)carbamate

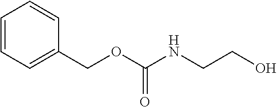 [SMILES: O=C(NCCO)OCc1ccccc1]

The title compound was prepared according to the procedures described for the preparation of Intermediate 1 described above, or may also be purchased when available commercially.

Step 2. 2-(((benzyloxy)carbonyl)amino)ethyl(2-(trimethylammonio)ethyl)phosphate

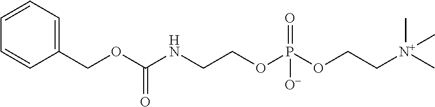 [SMILES: C[N+](C)(C)CCOP(=O)([O-])OCCNC(=O)OCc1ccccc1]

POCl₃ (57.5 mL, 1.2 eq.) was mixed with 500 mL of dichloromethane (DCM) in a 2-L round bottom flask. The POCl₃ solution was subsequently cooled to −20° C. or −15° C. In a separate flask, Et3N (107 mL, 1.5 eq.) and benzyl (2-hydroxyethyl)carbamate (100 g, 1 eq.) were mixed in DCM (500 mL). The resulting solution was then added to the POCl₃ solution, maintaining the temperature at −10° C. The reaction mixture was stirred at −10° C. for about one hour. While maintaining the temperature at −15° C., pyridine (165 mL, 4 eq.) was slowly added into the reaction mixture followed by the addition of choline tosylate (176.3 g, 1.25 eq.) The reaction mixture was stirred at −5° C. for one hour, after which the temperature was raised to 20° C. and stirred for about 12 hours. Water (300 mL) was added to the reaction mixture and the resulting biphasic solution was stirred at 20° C. for 6 hours before the DCM phase was separated. The aqueous phase was washed with DCM (20×80 mL) and concentrated under vacuum until oily residue was formed. The resulting residue was re-dissolved in 100 mL of solvent A (Water+0.1% TFA) and purified on SFAR C18 column 400 g on multiple runs of 120 mL each. The column was equilibrated with 100% A (Water+0.1% TFA) and 0% B (acetonitrile). Subsequent elution was performed in the following order: 100% A 6 column volumes (CV); from 0% B to 25% B in 14 CV; from 25% B to 100% B in 3 CV; 100% B isocratic for 4 CV. Fractions containing the product were collected and evaporated from the solvent to afford 2-(((benzyloxy)carbonyl)amino)ethyl(2-(trimethylammonio)ethyl)phosphate (˜50 g).

UPLC Purity: 86% (UPLC-MS generic method).

Step 3. 2-aminoethyl(2-(trimethylammonio)ethyl)phosphate (Intermediate 1)

2-(((benzyloxy)carbonyl)amino)ethyl(2-(trimethylammonio)ethyl)phosphate (30 g, 1 eq.) was dissolved in 30 mL of water. The solution was transferred in a 300-mL stainless steel hydrogenation vessel and was diluted with isopropyl alcohol (IPA, 200 mL). Palladium on charcoal 10% Pd/C (8.2 g) was added to the solution at about 20° C. The reaction mixture was then stirred under hydrogen pressure (7 bar) at constant temperature of about 20° C. for about 20 hours. The reaction mixture was then filtered on celite pad and the filtrate was washed with a solution of IPA/water (8:1). The resulting solution was concentrated under vacuum to yield an oily residue. The residue was re-dissolved in IPA (200 mL) and concentrated under vacuum. The re-dissolving step and the removal of the solvent were repeated once more to afford 2-aminoethyl(2-(trimethylammonio)ethyl)phosphate (18.8 g).

### Example 1. Preparation of 2-(5Z,8Z,11Z,14Z)-icosa-5,8,11,14-tetraenamidoethyl 2-(trimethylammonio) ethylphosphate (Compound 3)

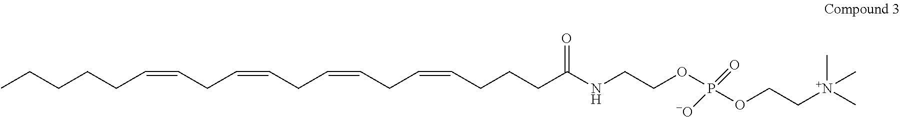

To a stirred solution of (5Z,8Z,11Z,14Z)-icosa-5,8,11,14-tetraenoic acid (2.5 g, 8.22 mmol, 1.0 eq.) in dimethylformamide (DMF, 15 mL) was added 1-[Bis(dimethylamino)methylene]-1H-1,2,3-triazolo[4,5-b]pyridinium 3-oxide hexafluorophosphate (HATU, 3.74 g, 9.86 mmol, 1.2 eq.) at room temperature. After stirring the reaction mixture for 30 minutes, N,N-Diisopropylethylamine (DIPEA, 4.29 mL, 24.6 mmol, 3.0 eq) was added followed by the addition of Intermediate 1 (2.78 g, 12.3 mmol, 1.5 eq) and the resulting mixture was stirred for about 18 hours at room temperature. The reaction mixture was diluted with isopropylether (IPE, 2×50 mL), cooled to 0° C., settled for 30 minutes, and the IPE layer was decanted (the diluting/decanting procedure was repeated a few times). The resulting residue was dried under vacuum. Isolated crude (8 g with 15% by LC-MS) was purified by PREP HPLC (Column: LUNA-C18, 250×21.2 mm, 1.6 μm; Mobile phase: [CH₃CN: Water+0.01% HCOOH]; B %: 80%-50%, 22 min.; 50%-90%, 35 min) to yield Compound 3 (1.6 g) as a light brown sticky solid. Mass [m/z]: 513.2 [M+H]⁺. Yield: 1.6 g (38.0%). LC-MS Purity: 96.0% (LCMS Method A). HPLC Method: 90.3% (Analytical HPLC method).

### Example 1A. Alternative Preparation of 2-(5Z,8Z,11Z,14Z)-icosa-5,8,11,14-tetraenamidoethyl 2-(trimethylammonio) ethylphosphate (Compound 3)

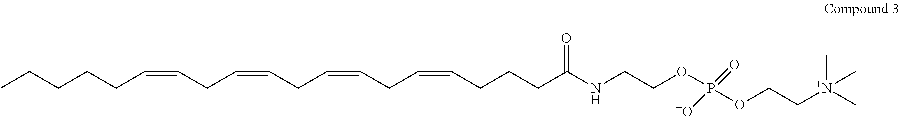

HATU (27.1 g, 1.2 eq.) was added to a stirred solution of (5Z,8Z,11Z,14Z)-icosa-5,8,11,14-tetraenoic acid (18.1 g, 1.0 eq.) in DMF (64 mL) at room temperature. After stirring the reaction mixture for about 30 minutes, DIPEA (31 mL, 3.0 eq) was added, followed by addition of Intermediate 1 (24.45 g, 1.2 eq.) solution in DMF (59 mL) and the resulting mixture was stirred for 18 hours at room temperature. The reaction mixture was then concentrated under reduced pressure to 2-3 vol of reaction mixture. n-Heptane (200 mL) was then added to the resulting residue and the obtained mixture was concentrated under vacuum until an oily residue was formed. A solvent swap with n-heptane was performed two times.

Water (150 mL) was then added to the residue and the resulting suspension was stirred for 30 minutes and then filtered. The obtained solution was concentrated under reduced pressure following the addition of n-BuOH. The resulting residue (about 60 mL) was purified by Biotage on 400 g SFAR C-18 column in a multiple runs. λ 210 nm; solvents: (A) water, (B) ACN; gradient: 3 CV isocratic 0% A; 10 CV from 0% to 50% B; isocratic 50% B for 3CV, 8 CV from 50% to 100% ACN; isocratic 100% B for 3 CV. Fractions containing products were collected and reduced via rotary evaporation (bath temp 20-25° C.) until ACN was removed before lyophilization to yield Compound 3. HPLC Method: 95.7% (Analytical HPLC method—40 min).

### Example 2. Alternative Synthesis of 2-(5Z,8Z,11Z,14Z)-icosa-5,8,11,14-tetraenamidoethyl 2-(trimethylammonio) ethylphosphate (Compound 3)

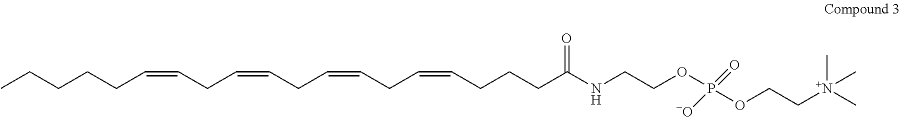

Step 1. 2,5-dioxopyrrolidin-1-yl (5Z,8Z,11Z,14Z)-icosa-5,8,11,14-tetraenoate

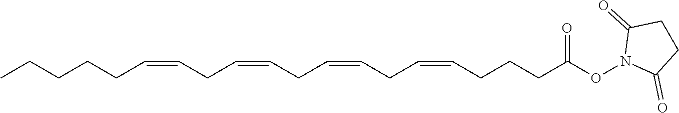 [SMILES: CCCCC/C=C\C/C=C\C/C=C\C/C=C\CCCC(=O)ON1C(=O)CCC1=O]

To a stirred solution of (5Z,8Z,11Z,14Z)-icosa-5,8,11,14-tetraenoic acid (5.0 g, 0.016 mol, 1.0 eq.) in ethyl acetate (EtOAc; 100 mL) was added N-hydroxysuccinimide (NHS; 2.07 g, 0.018 mmol, 1.1 eq.) at room temperature. After stirring the reaction mixture for about 10 minutes, N,N′-dicyclohexylcarbodiimide (DCC; 3.72 g, 0.018 mol, 1.1 eq.) was added and stirred for 18 hours at the same temperature. The reaction was monitored by TLC. The resulting reaction mixture was filtered through a celite pad and washed with EtOAc (2×15 mL). Collected filtrate was concentrated under reduced pressure to get crude residue (6.5 g) as brown color liquid, which was further purified by silica gel chromatography (60-120 mesh) and eluted with 5-8% of EtOAc/Hexane and concentrated under reduced pressure to yield 2,5-dioxopyrrolidin-1-yl (5Z,8Z,11Z,14Z)-icosa-5,8,11,14-tetraenoate (3.5 g) as a brown color liquid. Mass [m/z]: 402.3 [M+H]⁺. Yield: 3.5 g (53.1%). LC-MS Purity: ˜80%.

Step 2. 2-(5Z,8Z,11Z,14Z)-icosa-5,8,11,14-tetraenamidoethyl2-(trimethylammonio) ethylphosphate

To a stirred solution of 2,5-dioxopyrrolidin-1-yl (5Z,8Z,11Z,14Z)-icosa-5,8,11,14-tetraenoate (3.0 g, 7.4 mmol, 1.0 eq.) in DMF (30 mL) was added Et3N (2.1 mL, 14.8 mmol, 2.0 eq.) followed by the addition of Intermediate 1 (2.5 g, 11.1 mmol, 1.5 eq) at room temperature. The reaction mixture was further stirred for 18 hours at the same temperature. The reaction was monitored by TLC and LC-MS. The resulting reaction mixture was triturated with IPE (2×50 mL) twice to remove DMF and non-polar impurities, and dried under vacuum resulting crude residue (5.6 g with 93% by LC-MS). The material was further purified from flash chromatography (Column: Biotage-C18, 60 g Duo-100 Å 30 μm; Mobile phase: [ACN:Water+0.1% HCOOH]; B %: 5-30%, 0-20 min/30-45% 20-40 min.) to yield Compound 3 (1.5 g) as a light brown sticky solid. Mass [m/z]: 513.2 [M+H]*. Yield: 1.5 g (39.1%). LC-MS Purity: 97.5% (LCMS Method C). HPLC Purity: 91.6% (Analytical HPLC method).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims. It should be appreciated by those persons having ordinary skill in the art(s) to which the present invention relates that any of the features described herein in respect of any particular aspect and/or embodiment of the present invention can be combined with one or more of any of the other features of any other aspects and/or embodiments of the present invention described herein, with modifications as appropriate to ensure compatibility of the combinations. Such combinations are considered to be part of the present invention contemplated by this disclosure.

What is claimed is:

1. A process of preparing a compound of Formula I: 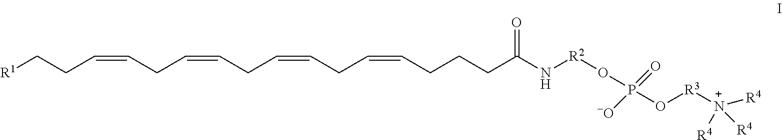
or a pharmaceutically acceptable salt thereof comprising reacting a compound of Formula II: 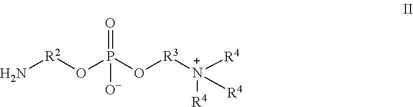
with a compound of Formula III: 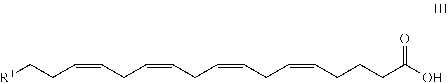
in the presence of a first base and an amine coupling agent, wherein:
R¹ is C₁₋₁₀ alkyl;
R² is C₁₋₆ alkylene;
R³ is C₁₋₆ alkylene; and
each R⁴ is independently selected from the group consisting of H and C₁₋₆ alkyl.

2. The process of claim 1, wherein the first base is an amine base.

3. The process of claim 1, wherein the first base is a tri(C₁₋₆ alkyl) amine base.

4. The process of claim 1, wherein the first base is N,N-diisopropylethylamine (DIPEA).

5. The process of any one of claims 1 to 4, wherein the amine coupling agent is 1-[bis(dimethylamino)methylene]-1H-1,2,3-triazolo[4,5-b]pyridinium 3-oxide hexafluorophosphate (HATU).

6. The process of any one of claims 1 to 5, wherein about 1 to about 5 equivalents of the compound of Formula II is used based on 1 equivalent of the compound of Formula III.

7. The process of any one of claims 1 to 6, wherein the reacting is performed at a temperature of from about 20° C. to about 30° C.

8. The process of any one of claims 1 to 7, wherein the reacting is performed in the presence of a first solvent component.

9. The process of claim 8, wherein the first solvent component comprises dimethylformamide.

10. The process of any one of claims 1 to 9, further comprising isolating the compound of Formula I, or a pharmaceutically acceptable salt thereof.

11. The process of claim 10, wherein the isolating comprises diluting the reaction mixture comprising the compound of Formula I, or a pharmaceutically acceptable salt thereof, with a fifth solvent component.

12. The process of claim 10 or 11, wherein the isolating comprises concentrating the reaction mixture comprising the compound of Formula I, or a pharmaceutically acceptable salt thereof, and performing a solvent swap with a fifth solvent component, thereby isolating the compound of Formula I, or a pharmaceutically acceptable salt thereof.

13. The process of claim 12, wherein the solvent swap is repeated one to four times.

14. The process of any one of claims 11 to 13, wherein the fifth solvent comprises isopropylether or n-heptane.

15. The process of any one of claims 1 to 14, wherein the compound of Formula II is prepared according to a process comprising deprotecting a compound of Formula IV: 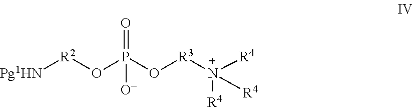
wherein Pg¹ is an amine protecting group.

16. The process of claim 15, wherein Pg¹ is benzyloxycarbonyl (Cbz).

17. The process of claim 15 or 16, wherein the deprotecting comprises reacting the compound of Formula IV with hydrogen gas in the presence of a hydrogenation catalyst.

18. The process of claim 17, wherein the hydrogenation catalyst comprises palladium on carbon.

19. The process of claim 17 or 18, wherein the pressure of the hydrogen gas is from about 1 bar to about 20 bar.

20. The process of any one of claims 15 to 19, wherein the deprotecting is performed at a temperature of from about 20° C. to about 30° C.

21. The process of any one of claims 15 to 20, wherein the deprotecting is performed in the presence of a second solvent component.

22. The process of claim 21, wherein the second solvent component comprises isopropanol.

23. The process of any one of claims 15 to 22, wherein the compound of Formula IV is prepared according to a process comprising:
i) reacting a compound of Formula V: 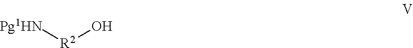
with phosphoryl trihalide in the presence of a second base to afford a first mixture; and
ii) reacting the first mixture with a compound of Formula VI: 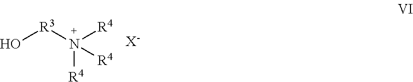
in the presence of a third base, wherein X⁻ is an anion.

24. The process of claim 23, wherein X⁻ is tosylate.

25. The process of claim 23 or 24, wherein the second base is an amine base.

26. The process of claim 23 or 24, wherein the second base is a tri(C₁₋₆ alkyl) amine base.

27. The process of claim 23 or 24, wherein the second base is triethylamine.

28. The process of any one of claims 23 to 27, wherein the reacting of step i) is performed at a temperature of from about −20° to about 25° C.

29. The process of any one of claims 23 to 28, wherein step i) is performed in the presence of a third solvent component.

30. The process of claim 29, wherein the third solvent component comprises chloroform.

31. The process of any one of claims 23 to 30, wherein the third base is an amine base.

32. The process of any one of claims 23 to 30, wherein the third base is pyridine.

33. The process of any one of claims 23 to 32, wherein step ii) is performed at a temperature of from about −10° C. to about 25° C.

34. The process of any one of claims 23 to 32, wherein step i) and step ii) are performed as a single pot reaction.

35. The process of any one of claims 23 to 34, wherein the compound of Formula V is prepared according to a process comprising reacting a compound of Formula VII:
Pg¹—X¹ VII
with a compound of Formula IX: 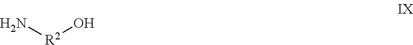
in the presence of a fourth base, wherein X¹ is halo.

36. The process of claim 35, wherein X¹ is chloro.

37. The process of claim 35 or 36, wherein the reacting is performed in the presence of a fourth solvent component.

38. The process of claim 37, wherein the fourth solvent component is dichloromethane.

39. The process of any one of claims 35 to 38, wherein the reacting is performed at a temperature of from about −10° C. to about 10° C.

40. The process of any one of claims 1 to 39, wherein R¹ is C₁₋₆ alkyl.

41. The process of any one of claims 1 to 39, wherein R¹ is propyl.

42. The process of any one of claims 1 to 41, wherein R² is ethylene.

43. The process of any one of claims 1 to 42, wherein R³ is ethylene.

44. The process of any one of claims 1 to 43, wherein each R⁴ is an independently selected C₁₋₆ alkyl.

45. The process of any one of claims 1 to 43, wherein each R⁴ is methyl.

46. The process of any one of claims 1 to 45, wherein the compound of Formula I is a compound of Formula Ia: 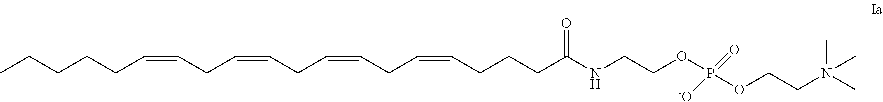
or a pharmaceutically acceptable salt thereof.

47. A process of preparing a compound of Formula Ia: 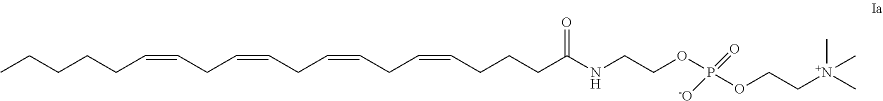
or a pharmaceutically acceptable salt thereof, comprising reacting a compound of Formula IIa: 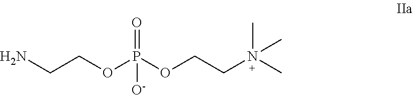
with a compound of Formula IIIa: 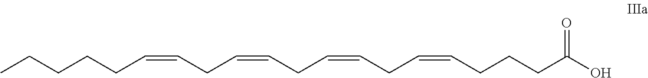 [SMILES: CCCCC/C=C\C/C=C\C/C=C\C/C=C\CCCC(=O)O]
in the presence of N,N-diisopropylethylamine and 1-[bis(dimethylamino)methylene]-1H-1,2,3-triazolo[4,5-b]pyridinium 3-oxide hexafluorophosphate (HATU).

48. The process of claim 47, further comprising isolating the compound of Formula Ia, or a pharmaceutically acceptable salt thereof.

49. The process of claim 48, wherein the isolating comprises diluting the reaction mixture comprising the compound of Formula Ia, or a pharmaceutically acceptable salt thereof, with a fifth solvent component.

50. The process of claim 48 or 49, wherein the isolating comprises concentrating the reaction mixture comprising the compound of Formula Ia, or a pharmaceutically acceptable salt thereof, and performing a solvent swap with a fifth solvent component, thereby isolating the compound of Formula Ia, or a pharmaceutically acceptable salt thereof.

51. The process of claim 50, wherein the solvent swap is repeated one to four times.

52. The process of any one of claims 48 to 51, wherein the fifth solvent comprises isopropylether or n-heptane.

53. A process of preparing a compound of Formula I: 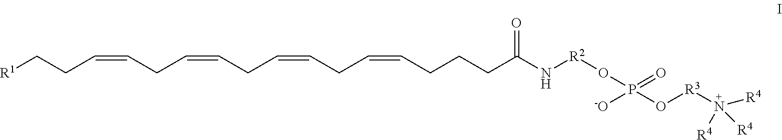
or a pharmaceutically acceptable salt thereof, comprising reacting a compound of Formula II: 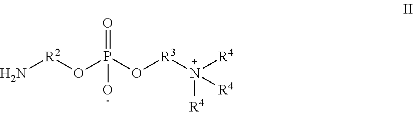
with a compound of Formula X: 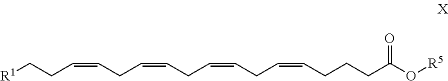
in the presence of a first base, wherein:
R¹ is C₁₋₁₀ alkyl;
R² is C₁₋₆ alkylene;
R³ is C₁₋₆ alkylene;
each R⁴ is independently selected from the group consisting of H and C₁₋₆ alkyl; and
R⁵ is a carboxylic acid activating group.

54. The process of claim 53, wherein the first base is an amine base.

55. The process of claim 53, wherein the first base is tri(C₁₋₆ alkyl) amine base.

56. The process of claim 53, wherein the first base is triethylamine.

57. The process of any one of claims 53 to 56, wherein R⁵ is 2,5-dioxopyrrolidin-1-yl.

58. The process of any one of claims 53 to 57, wherein about 1 to about 2 equivalents of the compound of Formula II is used based on 1 equivalent of the compound of Formula III.

59. The process of any one of claims 53 to 58, wherein the reacting is performed at a temperature of from about 20° C. to about 30° C.

60. The process of any one of claims 53 to 59, wherein the reacting is performed in the presence of a first solvent component.

61. The process of claim 60, wherein the first solvent component comprises dimethylformamide.

62. The process of any one of claims 53 to 61, wherein the compound of Formula X is prepared according to a process comprising reacting a compound of Formula XI: 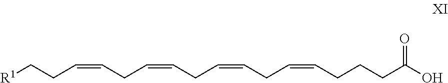
with a carboxylic acid activating agent in the presence of a O—N coupling agent.

63. The process of claim 62, wherein the carboxylic acid activating agent is N-hydroxysuccinimide (NHS).

64. The process of claim 62 or 63, wherein the O—N coupling agent is N,N′-dicyclohexylcarbodiimide (DCC).

65. The process of any one of claims 62 to 64, wherein the reacting is performed in the presence of a second solvent component.

66. The process of claim 65, wherein the second solvent component comprises ethyl acetate.

67. The process of any one of claims 53 to 66, wherein R¹ is C₁₋₆ alkyl.

68. The process of any one of claims 53 to 66, wherein R¹ is propyl.

69. The process of any one of claims 53 to 68, wherein R² is ethylene.

70. The process of any one of claims 53 to 69, wherein R³ is ethylene.

71. The process of any one of claims 53 to 70, wherein each R⁴ is an independently selected C₁₋₆ alkyl.

72. The process of any one of claims 53 to 70, wherein each R⁴ is methyl.

73. The process of any one of claims 53 to 72, wherein the compound of Formula I is a compound of Formula Ia: 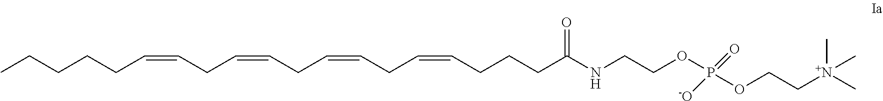
or a pharmaceutically acceptable salt thereof.

74. A process of preparing a compound of Formula Ia: 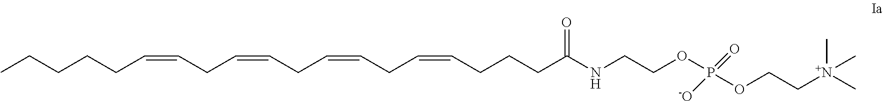
or a pharmaceutically acceptable salt thereof, comprising reacting a compound of Formula IIa: 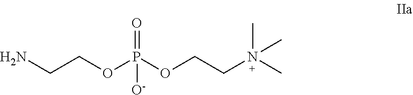
with a compound of Formula Xa: 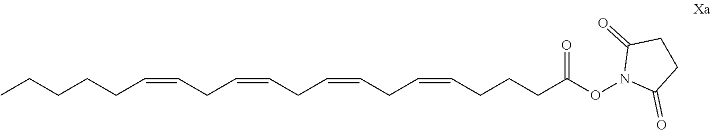 [SMILES: CCCCC/C=C\C/C=C\C/C=C\C/C=C\CCCC(=O)ON1C(=O)CCC1=O]
in the presence of triethylamine.

75. A compound of Formula Xa: 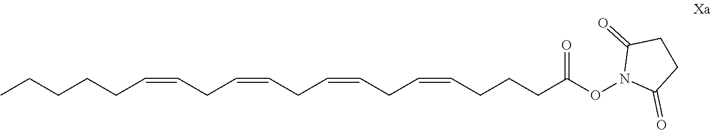 [SMILES: CCCCC/C=C\C/C=C\C/C=C\C/C=C\CCCC(=O)ON1C(=O)CCC1=O]
or a salt thereof.
